(19) United States
(12) Patent Application Publication
(10) Pub. No.: US 2016/0158530 A1
(43) Pub. Date: Jun. 9, 2016

(54) EXTRAVASCULAR IMPLANT TOOLS AND IMPLANT TECHNIQUES UTILIZING SUCH TOOLS

(71) Applicant: Medtronic, Inc., Minneapolis, MN (US)

(72) Inventors: Ronald A. DRAKE, St. Louis Park, MN (US); Kevin R. SEIFERT, Forest Lake, MN (US); Andrea J. ASLESON, Maple Grove, MN (US); Lester O. STENER, Hudson, WI (US); Nathan L. OLSON, Shoreview, MN (US)

(21) Appl. No.: 14/962,541

(22) Filed: Dec. 8, 2015

Related U.S. Application Data
(60) Provisional application No. 62/089,366, filed on Dec. 9, 2014.

(51) Int. Cl. A61N 1/05; A61N 1/39

(52) U.S. Cl. CPC A61N 1/05; A61N 1/3956

(57) ABSTRACT

This disclosure provides various embodiments of implant tools and implant techniques utilizing those tools to implant components within extravascular locations. In one example, an implant tool for implanting a component within an extravascular location of a patient comprise a handle and a shaft adjacent the handle. The shaft has a proximal end, a distal end, and a body formed to define an open channel that extends from near the proximal end to the distal end. The open channel has a first width. The body has at least one flexible portion that defines an opening via which the open channel is accessed. The opening has a second width that is less than the first width. In another example, a sheath with an opening having the second width may be placed on the shaft of the implant tool instead of the implant tool having the at least one flexible portion.

### TECHNICAL FIELD

The present disclosure relates to implant tools and techniques for implanting implantable medical leads or other implantable components in extravascular locations.

### BACKGROUND

Implantable cardiac defibrillator (ICD) systems are used to deliver high energy electrical pulses or shocks to a patient's heart to terminate life threatening arrhythmias, such as ventricular fibrillation. Traditional ICD systems include a housing that encloses a pulse generator and other electronics of the ICD and is implanted subcutaneously in the chest of the patient. The ICD is connected to one or more implantable medical electrical leads that are implanted within the heart, referred to herein as transvenous leads.

Traditional ICD systems that utilize transvenous leads may not be the preferable ICD system for all patients. For example, some patients with difficult vascular access precludes placement of transvenous leads. As another example, children and other younger patients may also be candidates for non-transvenous ICD systems. Moreover, transvenous leads may become fibrosed in the heart over time, making lead revision and extraction procedures challenging.

An extravascular ICD system may be preferred for these patients. An extravascular ICD system includes a lead (or leads) that are implanted extravascularly in the patient, e.g., outside and/or exclusive of the heart. As such, the extravascular ICD may eliminate the need to implant transvenous leads within the heart. Instead, the lead(s) may be implanted subcutaneously, substernally, or in other extravascular locations.

### SUMMARY

This disclosure provides various embodiments of implant tools and implant techniques utilizing those tools. In one example, a system for implanting an implantable component within an extravascular location of a patient includes an implant tool and a sheath. The implant tool that includes a handle and a shaft adjacent the handle. The shaft has a first length from a proximal end to a distal end, and an open channel that extends from near the proximal end to the distal end. The open channel has a width. The sheath is configured to be placed on the implant tool. The sheath includes a body having a second length from a proximal end and a distal, a channel formed by the body, the channel extending from the proximal end to the distal end of the body, the channel receiving the shaft of the implant tool when the sheath is placed on the implant tool, and an opening that extends along the body of the sheath from the proximal end to the distal end, wherein the channel is accessible via the opening and the opening is less than or equal to the width of the open channel of the shaft of the implant tool.

In another example, an implant tool for implanting a component within an extravascular location of a patient comprise a handle and a shaft adjacent the handle. The shaft has a proximal end, a distal end, and a body formed to define an open channel that extends from near the proximal end to the distal end. The open channel has a first width. The body has at least one flexible portion that defines an opening via which the open channel is accessed. The opening has a second width that is less than the first width.

This summary is intended to provide an overview of the subject matter described in this disclosure. It is not intended to provide an exclusive or exhaustive explanation of the apparatus and methods described in detail within the accompanying drawings and description below. Further details of one or more examples are set forth in the accompanying drawings and the description below.

### BRIEF DESCRIPTION OF THE DRAWINGS

FIG. 1A is a front view of an example extravascular ICD system implanted within a patient.

FIG. 1B is a side view of the extravascular ICD system implanted within the patient.

FIG. 1C is a transverse view of the extravascular ICD system implanted within the patient.

FIG. 2 is a conceptual diagram of another example extravascular ICD system implanted within a patient.

FIG. 3A illustrates an angled view of an example implant tool.

FIG. 3B illustrates a longitudinal side view of the example implant tool of FIG. 3A.

FIG. 3C illustrates a top view of a distal portion of a shaft of the example implant tool of FIG. 3A.

FIG. 3D illustrates a cross sectional view of a distal end of the example implant tool taken from A-A′ in FIG. 3B.

FIG. 4 is a schematic diagram illustrating an example delivery system that includes an implant tool and a sheath.

FIG. 5A illustrates a longitudinal side view of the delivery system of FIG. 4 with the sheath placed over the implant tool.

FIG. 5B illustrates a top view of the delivery system of FIG. 4 with the sheath placed over the implant tool.

FIG. 5C illustrates a cross sectional view of the delivery system of FIG. 4 with the sheath placed over the implant tool taken from A-A′ in FIG. 5A.

FIG. 6A illustrates a longitudinal side view of the sheath of the delivery system of FIG. 4.

FIG. 6B illustrates a top view of sheath of the delivery system of FIG. 4.

FIG. 6C illustrates a cross-section view of sheath of the delivery system of FIG. 4.

FIG. 6D illustrates an angled view of sheath of the delivery system of FIG. 4.

### DETAILED DESCRIPTION

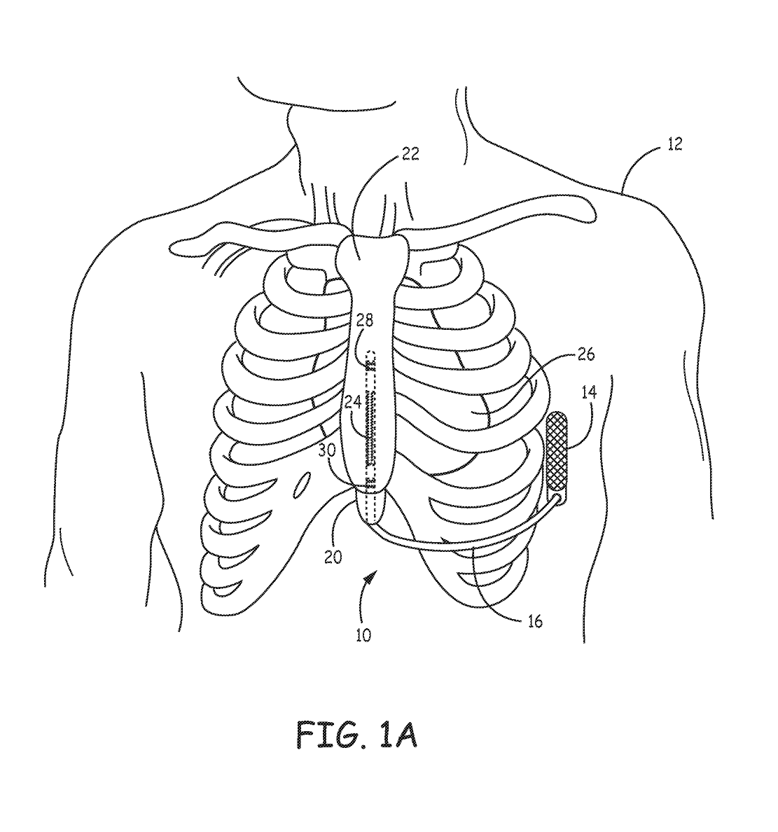

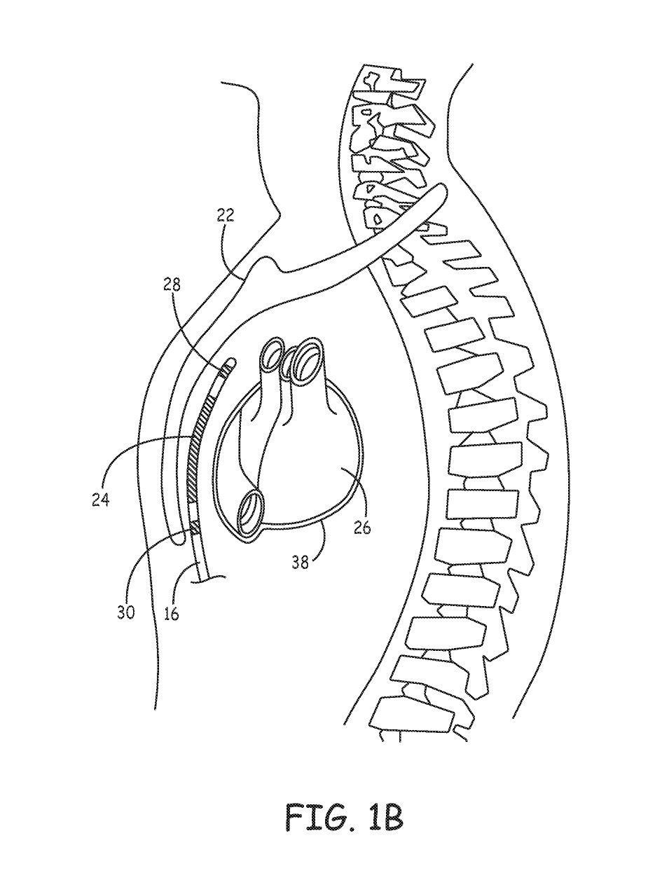

FIGS. 1A-C are conceptual diagrams of an extravascular ICD system 10 implanted within a patient 12. FIG. 1A is a front view of ICD system 10 implanted within patient 12. FIG. 1B is a side view of ICD system 10 implanted within patient 12. FIG. 1C is a transverse view of ICD system 10 implanted within patient 12. ICD system 10 includes an ICD 14 connected to a medical electrical lead 16. FIGS. 1A-C describe an implantable medical system capable of providing defibrillation and/or cardioversion shocks and, in some instances, pacing pulses. However, the techniques of this disclosure may also be used for implanting implantable medical leads, systems or devices configured to provide other electrical stimulation therapies to the heart or other organs, nerves, tissue or muscles (e.g., neurostimulators), or leads, catheters, devices or systems to provide other therapies (e.g., drug therapies).

ICD 14 may include a housing that forms a hermetic seal that protects components of ICD 14. The housing of ICD 14 may be formed of a conductive material, such as titanium, or of a combination of conductive and non-conductive materials. The conductive material of the housing functions as a housing electrode. ICD 14 may also include a connector assembly (also referred to as a connector block or header) that includes electrical feedthroughs through which electrical connections are made between lead 16 and electronic components included within the housing. The housing may house one or more processors, memories, transmitters, receivers, sensors, sensing circuitry, therapy circuitry, power sources and other appropriate components.

ICD 14 is configured to be implanted in a patient, such as patient 12. ICD 14 is implanted subcutaneously on the left midaxillary of patient 12. ICD 14 is on the left side of patient 12 above the ribcage. ICD 14 may, in some instances, be implanted between the left posterior axillary line and the left anterior axillary line of patient 12. ICD 14 may, however, be implanted at other subcutaneous locations on patient 12 such as at a pectoral location or abdominal location.

Lead 16 includes an elongated lead body having a proximal end that includes a connector (not shown) configured to be connected to ICD 14 and a distal portion that includes electrodes 24, 28, and 30. The implant tools and techniques of this disclosure may be used to implant lead 16 as described herein (or implant other types of leads, catheters, devices, or other implantable components). Lead 16 extends subcutaneously above the ribcage from ICD 14 toward a center of the torso of patient 12, e.g., toward xiphoid process 20 of patient 12. At a location near the center of the torso, lead 16 bends or turns and extends superior under/below sternum 22 within anterior mediastinum 36. Anterior mediastinum 36 may be viewed as being bounded laterally by pleurae 39, posteriorly by pericardium 38, and anteriorly by sternum 22. In some instances, the anterior wall of anterior mediastinum 36 may also be formed by the transversus thoracis and one or more costal cartilages. Anterior mediastinum 36 includes a quantity of loose connective tissue (such as areolar tissue), some lymph vessels, lymph glands, substernal musculature (e.g., transverse thoracic muscle), branches of the internal thoracic artery, and the internal thoracic vein. In one example, the distal portion of lead 16 may be implanted substantially within the loose connective tissue and/or substernal musculature of anterior mediastinum 36.

In other embodiments, the distal portion of lead 16 may be implanted in other non-vascular, extra-pericardial locations, including the gap, tissue, or other anatomical features around the perimeter of and adjacent to, but not attached to, the pericardium or other portion of heart 26 and not above sternum 22 or ribcage. As such, lead 16 may be implanted anywhere within the “substernal space” defined by the undersurface between the sternum and/or ribcage and the body cavity but not including the pericardium or other portion of heart 26. The substernal space may alternatively be referred to by the terms “retrosternal space” or “mediastinum” or “infrasternal” as is known to those skilled in the art and includes the anterior mediastinum 36. The substernal space may also include the anatomical region described in Baudoin, Y. P., et al., entitled “The superior epigastric artery does not pass through Larrey's space (trigonum sternocostale).” Surg. Radiol. Anat. 25.3-4 (2003): 259-62. In other words, the distal portion of lead 16 may be implanted in the region around the outer surface of heart 26, but not attached to heart 26.

The distal portion of lead 16 may be implanted substantially within anterior mediastinum 36 such that electrodes 24, 28, and 30 are located near a ventricle of heart 26. For instance, lead 16 may be implanted within anterior mediastinum 36 such that electrode 24 is located over a cardiac silhouette of one or both ventricles as observed via an anterior-posterior (AP) fluoroscopic view of heart 26. In one example, lead 16 may be implanted such that a therapy vector from electrode 24 to a housing electrode of ICD 14 is substantially across the ventricles of heart 26. The therapy vector may be viewed as a line that extends from a point on electrode 24, e.g., center of electrode 24, to a point on the housing electrode of ICD 14, e.g., center of the housing electrode. However, lead 16 may be positioned at other locations as long as the therapy vector between electrode 24 and the housing electrode is capable of defibrillating heart 26.

In the example illustrated in FIGS. 1A-C, lead 16 is located substantially centered under sternum 22. In other instances, however, lead 16 may be implanted such that it is offset laterally from the center of sternum 22. In some instances, lead 16 may extend laterally enough such that all or a portion of lead 16 is underneath/below the ribcage in addition to or instead of sternum 22.

The elongated lead body of lead 16 contains one or more elongated electrical conductors (not illustrated) that extend within the lead body from the connector at the proximal lead end to electrodes 24, 28, and 30 located along the distal portion of lead 16. The elongated lead body may have a generally uniform shape along the length of the lead body. In one example, the elongated lead body may have a generally tubular or cylindrical shape along the length of the lead body. The elongated lead body may have a diameter of between 3 and 9 French (Fr) in some instances. However, lead bodies of less than 3 Fr and more than 9 Fr may also be utilized. In another example, the distal portion (or all of) the elongated lead body may have a flat, ribbon or paddle shape. In this instance, the width across the flat portion of the flat, ribbon or paddle shape may be between 1 and 3.5 mm. Other lead body designs may be used without departing from the scope of this disclosure. The lead body of lead 16 may be formed from a non-conductive material, including silicone, polyurethane, fluoropolymers, mixtures thereof, and other appropriate materials, and shaped to form one or more lumens within which the one or more conductors extend. However, the techniques are not limited to such constructions.

The one or more elongated electrical conductors contained within the lead body of lead 16 may engage with respective ones of electrodes 24, 28, and 30. In one example, each of electrodes 24, 28, and 30 is electrically coupled to a respective conductor within the lead body. The respective conductors may electrically couple to circuitry, such as a therapy module or a sensing module, of ICD 14 via connections in connector assembly, including associated feedthroughs. The electrical conductors transmit therapy from a therapy module within ICD 14 to one or more of electrodes 24, 28, and 30 and transmit sensed electrical signals from one or more of electrodes 24, 28, and 30 to the sensing module within ICD 14.

Defibrillation electrode 24 is illustrated in FIG. 1 as being an elongated coil electrode. Defibrillation electrode 24 may vary in length depending on a number of variables. Defibrillation electrode 24 may, in one example, have a length between approximately 5-10 centimeters (cm). However, defibrillation electrode 24 may have a length less than 5 cm and greater than 10 cm in other embodiments. Another example, defibrillation electrode 24 may have a length between approximately 2-16 cm.

In other embodiments, however, defibrillation electrode 24 may be a flat ribbon electrode, paddle electrode, braided or woven electrode, mesh electrode, segmented electrode, directional electrode, patch electrode or other type of electrode besides an elongated coil electrode. In one example, defibrillation electrode 24 may be formed of a first segment and a second segment separated by a distance and having an electrode or a pair of electrodes (such as electrode 28 and/or 30 described below) located between the first and second defibrillation electrode segments. In one example, the segments may be coupled to the same conductor within the lead body such that the first and second segments function as a single defibrillation electrode. In other embodiments, defibrillation lead 16 may include more than one defibrillation electrode. For example, the first and second segments described above may be coupled to different conductors within the lead body such that the first and second segments function as separate defibrillation electrodes along the distal portion of lead 16. As another example, defibrillation lead 16 may include a second defibrillation electrode (e.g., second elongated coil electrode) near a proximal end of lead 16 or near a middle portion of lead 16.

Lead 16 also includes electrodes 28 and 30 located along the distal portion of lead 16. In the example illustrated in FIGS. 1A-C, electrode 28 and 30 are separated from one another by defibrillation electrode 24. In other examples, however, electrodes 28 and 30 may be both distal of defibrillation electrode 24 or both proximal of defibrillation electrode 24. In instances in which defibrillation electrode 24 is a segmented electrode with two defibrillation segments, electrodes 28 and 30 may be located between the two segments. Alternatively, one of electrodes 28 and 30 may be located between the two segments with the other electrode located proximal or distal to defibrillation electrode 24. Electrodes 28 and 30 may comprise ring electrodes, short coil electrodes, hemispherical electrodes, segmented electrodes, directional electrodes, or the like. Electrodes 28 and 30 of lead 16 may have substantially the same outer diameter as the lead body. In one example, electrodes 28 and 30 may have surface areas between 1.6-55 mm². Electrodes 28 and 30 may, in some instances, have relatively the same surface area or different surface areas. Depending on the configuration of lead 16, electrodes 28 and 30 may be spaced apart by the length of defibrillation electrode 24 plus some insulated length on each side of defibrillation electrode, e.g., approximately 2-16 cm. In other instances, such as when electrodes 28 and 30 are between a segmented defibrillation electrode, the electrode spacing may be smaller, e.g., less than 2 cm or less the 1 cm. The example dimensions provided above are exemplary in nature and should not be considered limiting of the embodiments described herein. In other examples, lead 16 may include a single pace/sense electrode or more than two pace/sense electrodes.

In some instances, electrodes 28 and 30 of lead 16 may be shaped, oriented, designed or otherwise configured to reduce extracardiac stimulation. For example, electrodes 28 and 30 of lead 16 may be shaped, oriented, designed or otherwise configured to focus, direct or point electrodes 28 and 30 toward heart 26. In this manner, pacing pulses delivered via lead 16 are directed toward heart 26 and not outward toward skeletal muscle. For example, electrodes 28 and 30 of lead 16 may be partially coated or masked with a polymer (e.g., polyurethane) or another coating material (e.g., tantalum pentoxide) on one side or in different regions so as to direct the pacing signal toward heart 26 and not outward toward skeletal muscle.

ICD 14 may obtain sensed electrical signals corresponding with electrical activity of heart 26 via a combination of sensing vectors that include combinations of electrodes 28 and/or 30 and the housing electrode of ICD 14. For example, ICD 14 may obtain electrical signals sensed using a sensing vector between electrodes 28 and 30, obtain electrical signals sensed using a sensing vector between electrode 28 and the conductive housing electrode of ICD 14, obtain electrical signals sensed using a sensing vector between electrode 30 and the conductive housing electrode of ICD 14, or a combination thereof. In some instances, ICD 14 may even obtain sensed electrical signals using a sensing vector that includes defibrillation electrode 24.

ICD 14 analyzes the sensed electrical signals obtained from one or more of the sensing vectors of lead 16 to monitor for tachyarrhythmia, such as ventricular tachycardia or ventricular fibrillation. ICD 14 generates and delivers substernal electrical stimulation therapy, e.g., ATP, cardioversion or defibrillation shocks, and/or post-shock pacing in response to detecting tachycardia (e.g., VT or VF). In some instances, ICD 14 may generate and deliver bradycardia pacing in addition to ATP, cardioversion or defibrillation shocks, and/or post-shock pacing.

In the example illustrated in FIG. 1, system 10 is an ICD system that provides cardioversion/defibrillation and/or pacing therapy. However, the implant tools and techniques may be utilized to implant other types of implantable medical leads, catheters (e.g., drug delivery catheters), or other implantable component or assembly. In addition, it should be noted that system 10 may not be limited to treatment of a human patient. In alternative examples, system 10 may be implemented in non-human patients, e.g., primates, canines, equines, pigs, ovines, bovines, and felines. These other animals may undergo clinical or research therapies that may benefit from the subject matter of this disclosure.

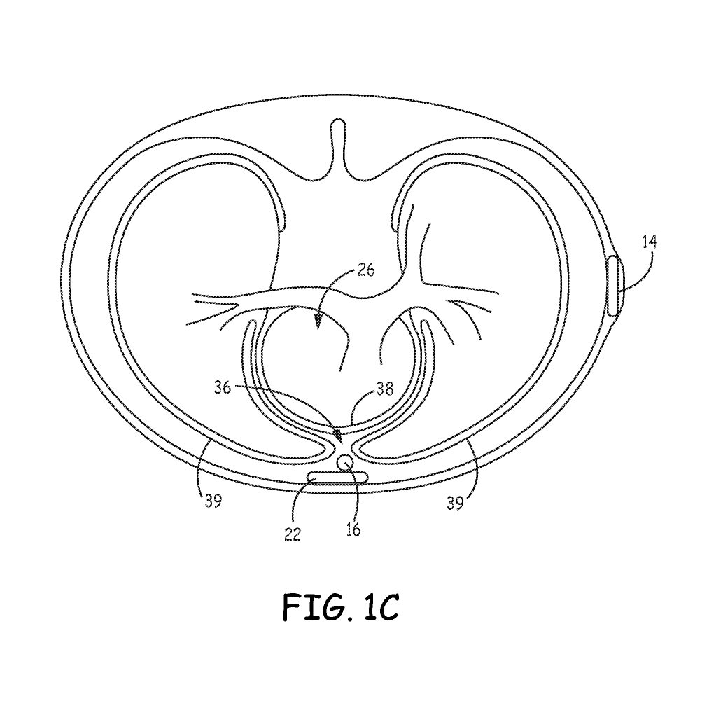

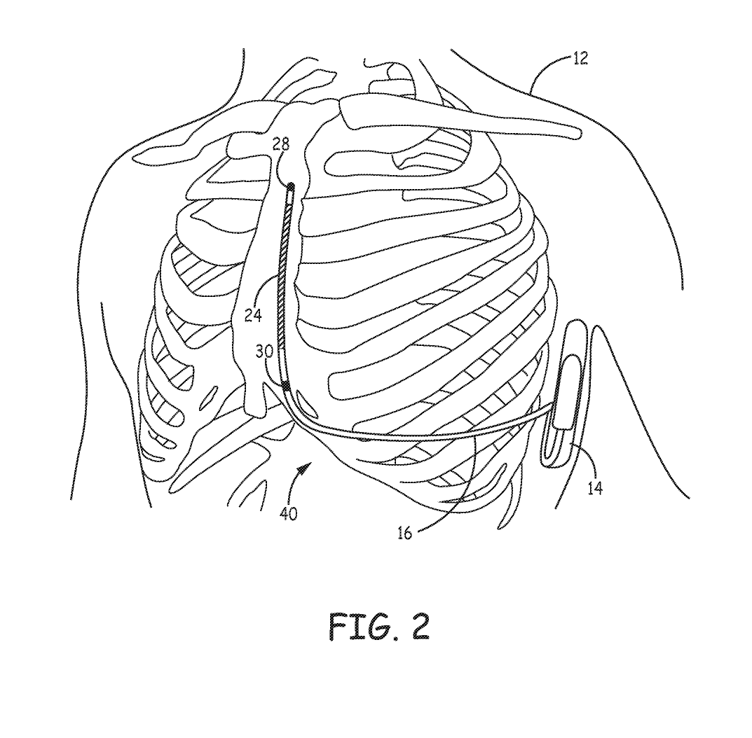

FIG. 2 is a conceptual diagram of another example extravascular ICD system 40 implanted within patient 12. In the example illustrated in FIG. 2, extravascular ICD system 40 is an implanted subcutaneous ICD system. ICD system 40 conforms substantially to ICD system 10 of FIGS. 1A-1C except that the distal portion of lead 16 is implanted subcutaneously above the sternum and/or the ribcage. In this case, ICD 14 may include additional components necessary to generate high voltage shocks at energies greater than ICD system 10, e.g., up to 80 J in the case of a subcutaneous ICD system 40 instead of 35-60 J in the case of the substernal ICD system 10.

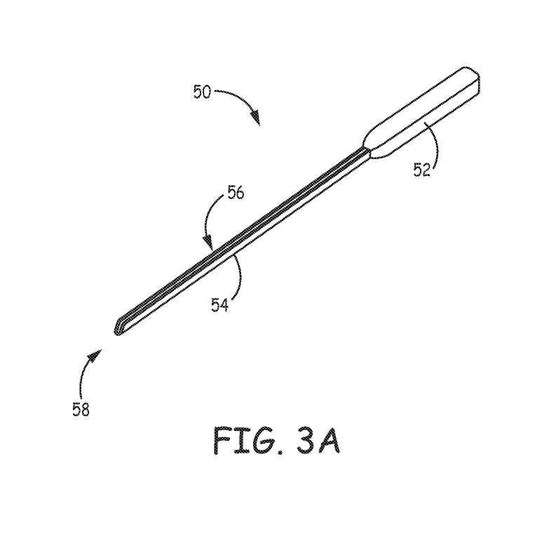

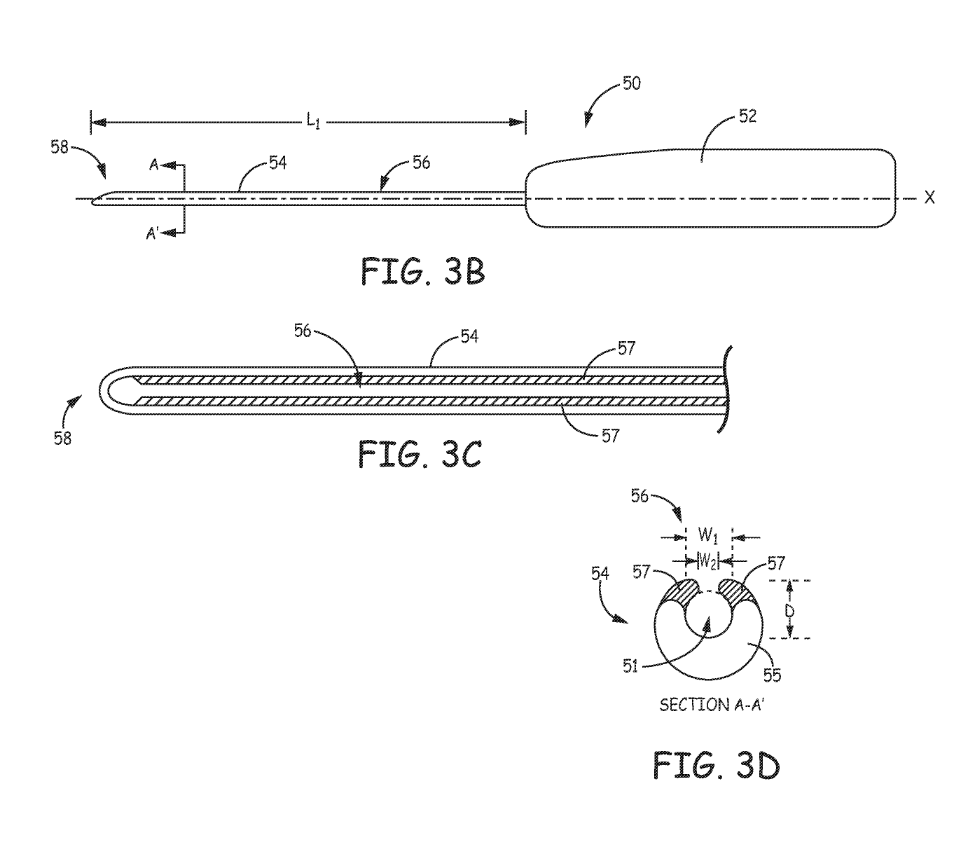

FIGS. 3A-D are conceptual drawings illustrating an example implant tool 50 for implanting a medical lead, such as lead 16 of FIGS. 1 and 2, a catheter, or other implantable component within an extravascular location of the patient. FIG. 3A illustrates an angled view of implant tool 50. FIG. 3B illustrates a longitudinal side view of implant tool 50. FIG. 3C illustrates a top view of a distal portion of shaft 54 of implant tool 50. FIG. 3D illustrates a cross sectional view of a distal end of implant tool 50 taken from A-A′ in FIG. 3B. As will be described in further detail herein, implant tool 50 of FIGS. 3A-D may be particularly useful in implanting defibrillation lead 16 in patient 12 in a subcutaneous, substernal, or other extravascular location.

Implant tool 50 includes a handle 52 and an elongate shaft 54 adjacent to handle 52. A body of shaft 54 defines an open channel 51 that extends from near handle 52 to a distal end 58. Open channel 51 may, in some embodiments, extend the entire length of shaft 54 from handle 52 to distal end 58. Shaft 54 has a length, labeled “L1” in FIG. 3B. The length L1 of shaft 54 may be determined based on the desired tunneling application. For example, shaft 54 may have a length between approximately 5 to 11 inches in some instances. However, other lengths may be appropriate for other desired applications.

Shaft 54 may have a relatively uniform thickness along the longitudinal length of shaft 54, e.g., along major axis “X” defined by implant tool 50. Alternatively, the thickness of the walls of shaft 54 may not be uniform along the length of shaft 54. For example, the walls of shaft 54 may have an increased thickness toward distal end 58 compared to the proximal end of shaft 54. The increase in thickness toward distal end 58 may enable improved tunneling performance by increasing rigidness or stiffness at distal end 58 or by reducing interference with the tissue. Additionally, the increase in thickness of distal end 58 may aid in shaping distal end to avoid coring, cutting, or puncturing of tissue, pleura, pericardium or other structures within patient 12. In other instances, distal end 58 and the proximal end near handle 52 of shaft 54 may have a greater thickness compared to the middle portion of shaft 54.

In some instances, shaft 54 may include markings (not shown) that may aid the user during the implant procedure. For example, the markings may be placed at locations on shaft 54 that coincide with features on lead 16 (e.g., electrodes, fixation mechanisms, or the like) when lead 16 is placed within open channel 51 such that the distal end of lead 16 is located at the distal end 58 of shaft 54. In instances in which the markings coincide with features of lead 16, the user may utilize the marking prior to beginning the procedure to place landmarks on the skin of patient 12. For example, prior to creating incisions, the user may place implant tool 50 on the skin of the patient such that the markings of the shaft coinciding with a desired location of the electrodes 18, 20 and 22 of lead 16. The user may then place landmarks on the skin of patient 12, such as landmarks corresponding with a desired end point of a tunnel or a desired tunneling path that places the features (e.g., electrodes 18, 20, and 22) of lead 16 at the desired location. In this manner, the user may use the markings on the shaft of implant tool 50 to be more confident that when insertion tool 50 is routed according to the landmarks on the skin that the electrodes or other lead features will be in the desired locations. The markings may additionally or alternatively provide the user feedback regarding the distance tunneled, in which case the markings may be located toward the proximal end of shaft 54. The markings may be laser etched, printed, or otherwise placed on shaft 54 of implant tool 50. The markings may be made within open channel 51 and/or on the outer surface of shaft 54.

As illustrated in the cross sectional view of distal end 58 of shaft 54, taken perpendicular to the longitudinal length of shaft 54 from handle 52 to distal end 58 (e.g., orthogonal to the major axis X defined by implant tool 50), shaft 54 has a generally C-shaped cross section that defines a generally C-shaped open channel 51. In other examples, however, the cross-section of shaft 54 and open channel 51 may be formed into any of a number of different shapes including, but not limited to, a U-shape, horseshoe-shape, arc-shape, or other shape.

Open channel 51 has a depth, labeled “D” in FIG. 3D. Depth D of channel 51 may, in one example, be approximately equal to an outer diameter the lead. In further examples, the depth D of open channel 51 may be slightly larger than the outer diameter of the lead to provide some margin. In further instances, open channel 51 may be sized to account for the largest portion of the lead, such as a fixation mechanism (such as tines), an anchoring sleeve, a connector, or other portion of the lead, with or without margin. The margin may allow the user push the lead along open channel 51 without too much interference or friction.

Open channel 51 also includes a width, labeled “W1” in FIG. 3D. In one example, width W1 of open channel 51 is approximately equal to the outer diameter of the lead such that when the implantable electrical lead 16 is placed within open channel 51 there is a slight interference fit. In another example, width W1 of open channel 51 is greater than an outer diameter of the lead (e.g., the diameter of the lead plus a slight margin).

Implant tool 50 includes flexible portions 57 that form an opening 56 to access open channel 51. In some instances, flexible portions 57 extend along the length L1 of shaft 54. In other instances, flexible portions 57 extend only along a portion of the length L1 of shaft 54, e.g., along only a distal portion of the length of the shaft.

The ends of flexible portions 57 may be separated from one another by a gap to form opening 56. The width of opening 56 formed by the ends of flexible portions 57, labeled “W2” in FIG. 3D, is less than the width W1 of open channel 51. Thus, the width W2 of opening 56 formed by flexible portions 57 is less than the outer diameter of the lead, catheter, or other component implant tool 50 is designed to implant in one example. Opening 56 may vary in size depending upon the desired application. In one example, the width W2 of opening 56 may be at least approximately 10% less than width W1. In another example, the width W2 of opening 56 may be approximately 10% less than the width of lead 16. However, in other examples, opening 56 may be less than 10% less than the width W1 or the diameter of lead 16 or more than 10% less than the width W1 or the diameter of lead 16. In other instances, the width W2 of opening 56 may be at least approximately 25% less than width W1 or the diameter of lead 16. Opening 76 may be larger or smaller than illustrated in FIG. 3D. In yet another example, opening 56 defined by the ends of flexible portions 57 may not define a gap. Instead, the ends of flexible portions 57 may be in contact with one another or overlapping such that opening 56 does not define a gap, but the ends of flexible portions 57 are not mechanically coupled and are moveable relative to one another such that open channel 51 is still accessible. Flexible portions 57 of shaft 54 flex outward to allow the lead to be placed within the open channel 51. Flexible portions 57 function to keep the lead in place once the lead is inserted into open channel 51.

Opening 56 may have a uniform width W2 along the length of shaft 54. Alternatively, width W2 of opening 56 may vary along the length of shaft 54. In one example, the width of opening 56 toward a proximal end of shaft 54 may be wider than the width of opening 56 toward a distal end of shaft 54 so that a user of implant tool 50 may more easily access open channel 51 at the proximal end. This may allow the user to more easily feed the lead 16 (or other implantable component) into the open channel 51 when the remainder of shaft 54 is within the patient.

Although implant tool 50 is illustrated as including two flexible portions 57, in another example, implant tool 50 may include only a single flexible portion 57. In this case, an opening may be formed between the single flexible portion 57 and the remainder 55 of the body of shaft 54. The single flexible portion would permit access to open channel 51 while still aiding in retaining the lead or other implantable component within open channel 51.

Shaft 54 may have the same cross section along the entire length of shaft 54. Alternatively, shaft 54 may have varying cross sections along portions of the length of shaft 54. For example, shaft 54 may have a more open cross-section, e.g., a U-shaped cross-section toward a proximal end of shaft 54 and more closed cross-section, e.g., a C-shaped cross-section along the mid and distal sections of shaft 54. Other varying cross-sections may be utilized without departing from the scope of this disclosure.

In the examples described above, implant tool 50 may be to be used to implant a particular sized lead such that a different implant tool or interchangeable shaft (e.g., having a different sized open channel 51) may be selected depending on the size of the lead to be implanted, which may range from 2 French to 11 French. In further examples, a single implant tool 50 may be designed to deliver leads having a variety of different diameters. In this case, the depth D and width W of open channel 51 may be sized for delivery of the largest diameter lead for which tool 50 is designed.

Shaft 54 may have a relatively uniform thickness along the sides and bottom of the body of shaft 54. In other words, the walls along the sides and bottom of shaft 54 may all have about the same thickness. In another example, however, shaft 54 may have thicker walls along the sides of shaft 54 forming open channel 51 than along the bottom of shaft 54.

Elongate shaft 54 of implant tool 50 is formed such that it is stiff enough to be capable of being pushed through the tissue, muscle or other structure to form a path through the body. Flexible portion 57 may be made of a polymer, copolymer, thermal plastic elastomer (TPE), or other material or combinations of material. In one example, flexible portions 57 are made from a softer, weaker polymer or TPE to allow them to flex when placing the lead within or removing the lead from open channel 51, while the remainder 55 of shaft 54 is made from a material that is more rigid than flexible portions 57 (e.g., metal or a rigid polymer). In one example, flexible portions 57 may be constructed of a material having a Young's Modulus of less than approximately 0.5 gigapascals (GPa) while the remainder 55 of shaft 54 may be constructed of a material having a Young's Modulus of greater than 3 GPa. In another example, flexible portions 57 may be constructed of a material having a Young's Modulus of between 0.01-0.1 GPa while the remainder 55 of shaft 54 may be constructed of a material having a Young's Modulus of between 3-200 GPa. Of course, the ability to flex is also a function of relative wall thickness and overall shape. Such a tool could be extruded, molded, or inserted as part of a manufacturing process and would provide additional stiffness and malleability to the implant tool.

In some instances, such as when shaft 54 is made of metal or a combination of metal and polymer, shaft 54 may be malleable. In other instances, shaft 54 of tool 50 may not be malleable, e.g., when shaft 54 is made of a molded polymer. In further instances, the implant tool may include a pre-formed or pre-shaped shaft 54. In this case, shaft 54 may be somewhat flexible while still being stiff enough to tunnel through tissue. The flexibility may allow a user to manipulate the tool slightly to control direction (e.g., steer) of the tunnel.

Handle 52 of implant tool 50 may also be made of a metal, alloy, polymer, or other material or combination of materials. Handle 52 and elongate shaft 54 may, in some instances, be constructed of the same material. For example, implant tool 50 may be formed of a single, unitary piece of material, such as metal or rigid polymer. In other instances, handle 52 and elongate shaft 54 may be constructed of different materials. In this case, handle 52 and shaft 54 may be formed of separate components that are attached together to form implant tool 50, e.g., via a two piece construction. For example, handle 52 may be made of polymer and shaft 54 may be made as described above and attached to handle 52 to form implant tool 50. Example metals or alloys from which handle 52 or rigid portions of shaft 54 may be constructed include, but are not limited to, stainless steel, titanium, titanium alloys, nickel-cobalt, and nickel-cobalt alloys. Example polymers may include, but are not limited to, acetal resin (e.g., DELRIN®), polyether ether ketone (PEEK), polycarbonate, polypropylene composites, and liquid-crystal polymer (LCP). In addition, lubricious fillers and coatings may be used to improve lubricity during tunneling and lead insertion. Such additives or coatings include, but are not limited to, siloxane, PTFE, and Foster ProPell™. Further, one or more additives or materials may be added to shaft 54 to make the shaft 54 or portions or shaft 54 radiopaque. For example, one or more radiopaque additives, which may include, without limitation, BaSO4, WC, and Bi2O3, may be added to shaft 54. As another example, a wire or other structure may be added to a polymer shaft for radiopacity.

Distal end 58 of shaft 54 may be shaped to aid in tunneling through tissue or muscle. For example, distal end 58 of the shaft 54 may be tapered, angled, blunt, rounded, pointed, bent or otherwise shaped to enable a user to tunnel through subcutaneous tissue without excess damage to surrounding tissue, piercing through the skin, or coring of the tissue.

A user of tool 50 may insert tool 50 into an incision and tunnel distal end 58 of shaft 54 to a desired location. Once at the desired location, the user may deliver an implantable electrical lead, such as defibrillation lead 16 of FIG. 1, catheter or other implantable structure in the tunnel or path formed by implant tool 50 by pushing the defibrillation lead 16 through open channel 51 of shaft 54 and then removing tool 50 while leaving defibrillation lead 16 in the path created by the implant tool. In other instances, the implantable electrical lead 16 may be placed within open channel 51 prior to tunneling through the tissue or muscle such that the tunneling of the path and placement of lead 16 within the path occurs concurrently.

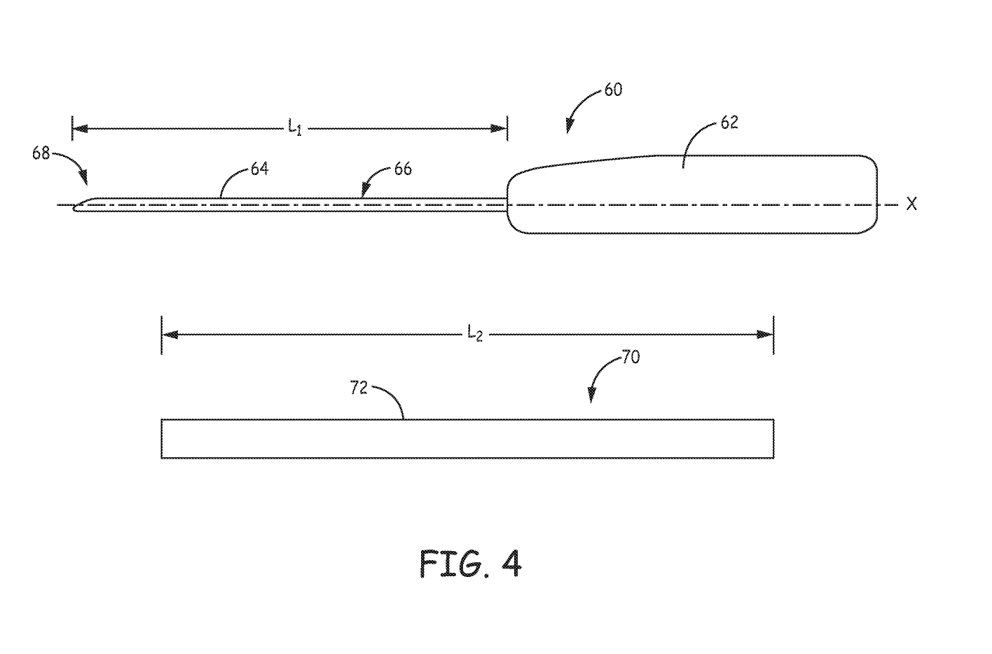

FIG. 4 is a schematic diagram illustrating another delivery system that includes an implant tool 60 and a sheath 70. Implant tool 60 includes a handle 62, a shaft 64, an open channel 66, and a distal end 68. Handle 62, shaft 64, open channel 66, and distal end 68 of implant tool 60 may conform substantially to handle 52, shaft 54, open channel 51 and distal end 58 of implant tool 50 described above with respect to FIGS. 3A-3D, but shaft 64 of implant tool 60 does not include flexible portions 57. Shaft 64 of FIG. 4 has a cross-section that is more semi-circle -shaped than C-shaped. The description of FIGS. 3A-3D will not be repeated here for sake of brevity, but the like features may include similar structure and function described in FIGS. 3A-3D.

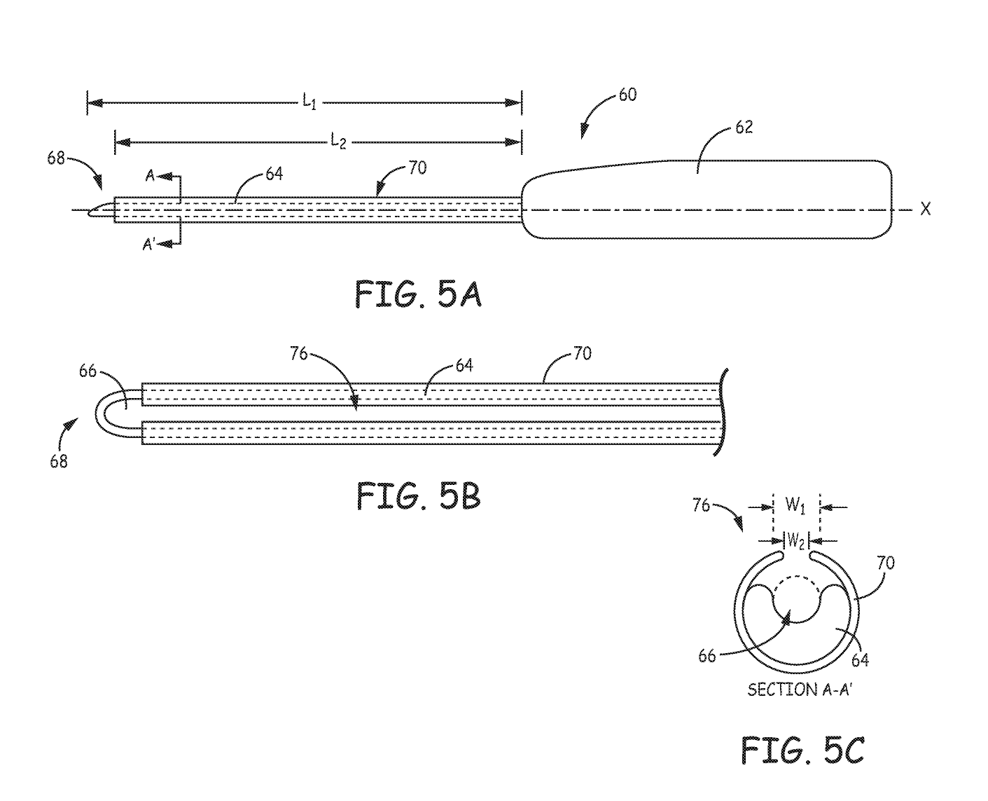

To reduce the likelihood of a lead (or other implantable component) from prematurely exiting open channel 51, open channel sheath 70 is placed over shaft 64 of implant tool 60 as will be illustrated in FIGS. 5A-C. This provides a similar function to that described above for flexible portions 57 of FIGS. 3A-3D. FIG. 5A illustrates a longitudinal side view of the delivery system with sheath 70 placed over implant tool 60. FIG. 5B illustrates a top view of the delivery system with sheath 70 placed over implant tool 60. FIG. 5C illustrates a cross sectional view of the delivery system with sheath 70 placed over implant tool 60 taken from A-A′ in FIG. 5A.

Sheath 70 has a length (L2) that is less than the length (L1) of shaft 64 of implant tool 60. As such, when sheath 70 is placed over shaft 64 of implant tool 60, shaft 60 extends from the distal end of sheath 70. As illustrated best in FIG. 5C, the arc length of sheath 70 is greater than the arc length of shaft 64 such that the ends of the body of sheath 70 that form opening extend over open channel 66 to aid in holding a lead or other component within open channel 66. Thus, at least a portion of the body of sheath 70 extends over open channel 66 of shaft 64 when opening 76 of sheath 70 is aligned with open channel 66 of shaft 64. Opening 76 of sheath 70 is less than or equal to the width of open channel 66 of the shaft 64 of the implant tool 60. In one example, opening 76 of sheath 70 is at least ten percent (10%) less than the width of open channel 66 of shaft 64 of the implant tool 60. In another example, opening 76 of sheath 70 is at least twenty five percent (25%) less than the width of open channel 66 of shaft 64 of the implant tool 60. When sheath 70 is placed on shaft 64 of implant tool 60, open channel 66 of shaft 64 is accessible via opening 76 of sheath 70.

Sheath 70 may also be moveable with respect to shaft 64. For example, sheath 70 may be rotated around the major axis “X” such that opening of sheath 70 may rotate around the body of shaft 64 of implant tool 60. For example, after placing lead 16 within open channel 66 via the opening 76 of sheath 70, sheath 70 may be rotated (e.g., 180 degrees around the major axis X) such that the lead may no longer exit opening 76. When lead 16 is in place within the patient, sheath 70 may be rotated again (e.g., another 180 degrees) to allow lead 16 to exit opening 76 of sheath 70.

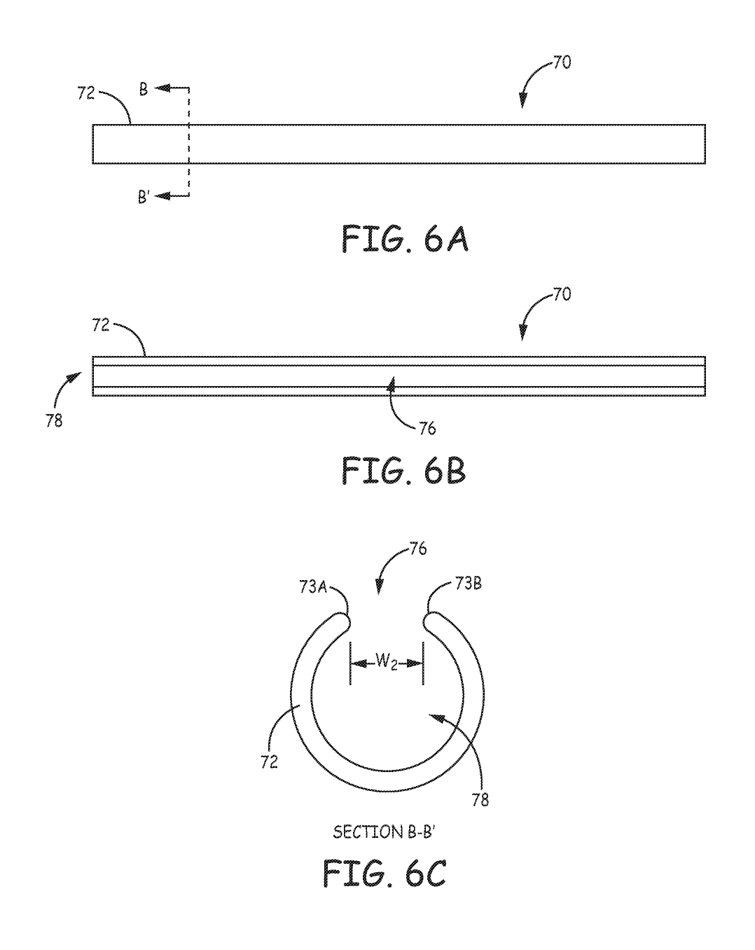

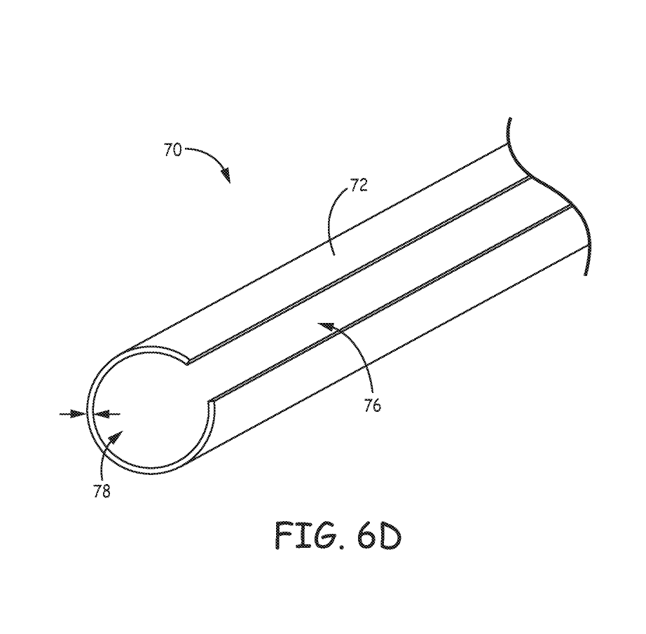

FIGS. 6A-6D illustrate various views of sheath 70 of FIG. 5 in further detail. FIG. 6A illustrates a longitudinal side view of sheath 70. FIG. 6B illustrates a top view of sheath 70. FIG. 6C illustrates a cross-section view of sheath 70. FIG. 6D illustrates an angled view of sheath 70.

Sheath 70 includes a body 72 having a proximal end and a distal end. In some instances, the distal end of body 72 may be tapered to aid in tunneling. Body 72 of sheath 70 defines an inner channel 78. In the examples described herein, the cross-section of an outside of body 72 and the inner channel 78 defined by body 72 is substantially C-shaped. However, the cross-section of either the outside of body 72 and/or the inner channel defined by body 72 may be a different shape depending on the desired application. The cross-section is taken normal (i.e., perpendicular) to the longitudinal length of sheath 70 from the distal end of body 72 to the proximal end of body 72.

Sheath 70 includes an opening 76 along the length of body 72. As described further herein, opening 76 along body 72 may form a gap between the ends of body 72 located at the boundary of the opening (as can be viewed in the cross-sectional view of sheath 70 in FIG. 6C). Inner channel 78 is accessible via opening 76. Opening 76 extends the entire length of body 72 from the distal end to the proximal end. In other examples, opening 76 may not extend the entire length of the body 72. Inner channel 78 is accessible via opening 76. In the example illustrated in FIGS. 6A-D, opening 76 follows a substantially straight path from the distal end of body 72 of sheath 70 to the proximal end of body 72 of sheath 70. In alternative configurations, however, opening 76 may follow other paths from the distal end of body 72 to the proximal end of body 72, such as spiral path, serpentine path, meandering path, or other path.

Sheath 70 may be sized such that sheath 70 fits on shaft 64 of implant tool 60 in such a manner that an interference fit is achieved between sheath 70 and shaft 64. The interference fit is achieved by friction after the parts are pushed together, rather than by any other means of fastening. The interference fit may, in some instances, be achieved by sizing and/or shaping the two mating parts so that one or the other, or both, slightly deviate in size from the nominal dimension. The interference fit may therefore be viewed as referring to the fact that one part slightly interferes with the space that the other is taking up. The tightness of the interference fit may be controlled by the amount of allowance, e.g., the planned difference from nominal size. Different allowances will result in various strengths of fit. The value of the allowance depends on which material is being used, how big the parts are, and what degree of tightness is desired.

In one example, the diameter of the inner channel formed by body 72 of sheath 70 may be equal to or slightly smaller than the outer diameter of shaft 64. The allowance in this case may be on the order of 1-10 thousandths of an inch. Allowances of less than 1 thousandth and greater than 10 thousands may be used, however. As such, when placed over shaft 64, sheath 70 slightly expands in diameter causing the interference fit. Other techniques for achieving an interference fit may also be utilized.

FIG. 6C illustrates a cross-sectional view of the distal end of sheath 70 taken from B-B′. As illustrated in FIG. 6C, body 72 is C-shaped such that opening 76 defines a gap between end 73A and end 73B of body 72. In other words, a gap exists along the circumference or cross-section of body 72. Opening 76 may have a width “W2.” Body 72 defines a channel 78 that extends along the length of body 72 from the distal end to the proximal end and through handle 74. In this case, channel 78 is a C-shaped channel, but the shape of channel 78 may vary depending on the cross-sectional shape of body 72. In some instances, opening 76 may have the same width W2 along the entire length of the body 72. In other instances, opening 76 at the proximal end of sheath 70 has a width that is larger than a width W1 at the distal end of body 72.

Sheath 70 may be formed to have a thickness that may vary depending the type of material used to form sheath 70, the desired rigidity of sheath 70, or the like. Sheath 70 should be rigid enough to not crumple, wrinkle, crease, or crush while being tunneled through tissue of patient 12. Sheath 70 may be made of extruded or molded material. The material may include, but not limited to, a polymer, a copolymer, a thermoplastic, or other material. Example materials include, but are not limited to, polyether block amide (such as PEBAX® 72D), polyether block amide blends (PEBAX® with a Foster ProPell™ additive), polyethylene, ultra-high-molecular-weight polyethylene (UHMWPE), Polytetrafluoroethylene (PTFE), nylons (such as GRILAMID® TR55 or L25, VESTAMID® L2140, AESNO®), or the like. In some instances, sheath 70 may be made from a material having a Young's Modulus in the same range as flexible portions 57, e.g., between approximately 0.01-0.1 GPa while shaft 64 of implant tool 60 may have a higher Young's Modulus, e.g., within the 3-200 GPa range. In some instances, sheath 70 may be made of multiple layers of different materials or may vary in materiality and durometer along the length of body 72. For example, sheath 70 may be formed of PEBAX® with a PTFE lining the inner surface of the channel. Other additives or coatings that may be applied to increase lubricity include, but are not limited to, siloxane, PTFE, and Foster ProPell™.

Opening 76 may vary in size depending upon the desired application. As described above, opening 76 of sheath 70 is less than or equal to the width of open channel 66 of the shaft 64 of the implant tool 60 and/or be less than the diameter of lead 16 that will be placed using sheath 70. In one example, opening 76 may be approximately 10% less than the width W1 of open channel 66 of the shaft 64 and/or approximately 10% less the diameter of lead 16. However, in other examples, opening 76 may be greater than or less than 10% of the width of open channel 66 of the shaft 64 and/or the diameter of lead 16 or more than 10% of the diameter of lead 16. Opening 76 may be larger or smaller than illustrated in FIGS. 6A-D.

The implant tools and/or systems described herein may be used to implant medical leads, catheters, or other implantable component. In one example, the implant tools and/or systems described herein may be used to implant a medical electrical lead at least partially within the substernal space, e.g., within anterior mediastinum of patient 12.

In one example, implant tool 50 is introduced into an incision near the center of the torso of patient 12. Implant tool 50 is advanced from the incision superior along the posterior of the sternum in the substernal space. The distal end of lead 16 (or other lead, catheter or implantable component) is introduced into open channel 51 of shaft 54 near the incision. As described above, the flexible portions 57 of shaft 54 flex to enable placement of lead 16 within open channel 51.

The distal end of defibrillation lead 16 is advanced along open channel 36 from the incision toward distal end 58 of shaft 54. As described above, flexible portions 57 of shaft 54 aid to hold lead 16 within open channel 66 as lead is advanced along open channel 36. Without flexible portions 57, lead 16 may pop out of open channel 36 while being advanced through the substernal space. Implant tool 50 is withdrawn toward the incision and removed from the body of patient 12 while leaving defibrillation lead 16 in place along the path along the posterior side of the sternum.

A similar technique may be performed using the delivery system formed by implant tool 60 and sheath 70. In this case, the implant tool 60 with the sheath placed over tool 70 may be advanced along the posterior side of the sternum. The lead 16 may be placed within channel 66 and sheath 70 may aid in holding lead 16 in place during advancement of lead 16 along channel 66.

Implant tool 50 (or the delivery system formed by implant tool 60 and sheath 70) may be used to form a subcutaneous tunnel lateral between the center of the torso of the patient to a pocket on the left side of the patient. Lead 16 may be advance along channel 51 (or channel 66) and implant tool 50 (or the delivery system formed by implant tool 60 and sheath 70) may be removed leaving the proximal portion of lead 16 in place along the lateral path. This may be done before or after the substernal tunneling. The connector of lead 16 may be connected to the ICD.

Various examples have been described. These and other examples are within the scope of the following claims.

1. A system for implanting an implantable component within an extravascular location of a patient, the delivery system comprising:
an implant tool that includes:
a handle, and
a shaft adjacent the handle, the shaft having a first length from a proximal end to a distal end, and an open channel that extends from near the proximal end to the distal end, the open channel having a width; and
a sheath configured to be placed on the implant tool, the sheath including:
a body having a second length from a proximal end and a distal,
a channel formed by the body, the channel extending from the proximal end to the distal end of the body, the channel receiving the shaft of the implant tool when the sheath is placed on the implant tool, and
an opening that extends along the body of the sheath from the proximal end to the distal end, wherein the channel is accessible via the opening and the opening is less than or equal to the width of the open channel of the shaft of the implant tool.

2. The system of claim 1, wherein, when the sheath is placed on the implant tool, the open channel of the shaft of the implant tool is accessible via the opening of the sheath.

3. The system of claim 1, wherein the opening of the sheath is at least ten percent (10%) less than the width of the open channel of the shaft of the implant tool.

4. The system of claim 1, wherein the sheath is made from at least one of a polymer, a copolymer, and a thermoplastic.

5. The system of claim 1, wherein the second length is less than the first length.

6. The system of claim 1, wherein
a cross-section of the shaft taken perpendicular to the first length of the shaft has a first arc length, and
a cross-section of the sheath taken perpendicular to the second length of the sheath has a second arc length, the second arc length being greater than the first arc length.

7. The system of claim 1, wherein at least a portion of the body of the sheath extends over the open channel of the shaft of the implant tool when the opening of the sheath is aligned with the open channel of the shaft of the implant tool.

8. The system of claim 1, wherein the sheath is rotatable with respect to the shaft of the implant tool.

9. The system of claim 1, wherein the opening of the sheath is wider at a proximal end of the sheath than at the distal end of the sheath.

10. The system of claim 1, wherein the sheath and the shaft form an interference fit that couples the sheath to the shaft.

11. An implant tool for implanting a component within an extravascular location of a patient, the implant tool comprising:
a handle, and
a shaft adjacent the handle, the shaft having:
a proximal end,
a distal end, and
a body formed to define an open channel that extends from near the proximal end to the distal end, the open channel having a first width, the body having at least one flexible portion that defines an opening via which the open channel is accessed, the opening having a second width that is less than the first width.

12. The implant tool of claim 11, wherein the second width is at least ten percent (10%) less than the first width.

13. The implant tool of claim 11, wherein the flexible portions of the body are made from at least one of a polymer, a copolymer, and a thermoplastic.

14. The implant tool of claim 13, wherein a remainder of the body is made from a material that is more rigid than the flexible portions of the body.

15. The implant tool of claim 11, wherein the flexible portions of the body are made from a material having a Young's Modulus of between 0.01-0.1 gigapascals (GPa) while a remainder of the body are made of a material having a Young's Modulus of between 3-200 GPa.

16. The implant tool of claim 11, wherein the shaft of the implant tool as a length that extends from the proximal end to the distal end and the flexible portions extend the entire length of the shaft.

17. The implant tool of claim 11, wherein the shaft of the implant tool as a length that extends from the proximal end to the distal end and the flexible portions extend only along a portion of the length of the shaft.

18. The implant tool of claim 17, wherein the flexible portions extend only along a distal portion of the length of the shaft.

19. The implant tool of claim 11, wherein the opening of the shaft is wider at a proximal end of the shaft than at the distal end of the shaft.

20. The delivery too of claim 11, wherein the body includes two flexible portions, the flexible portions defining the opening between ends of the flexible portions.

21. The implant tool of claim 11, wherein the body includes one flexible portion, the opening being defined between an end of the flexible portion and an end of a remainder of the body of the shaft.
